(19) United States
(12) Patent Application Publication
(10) Pub. No.: US 2024/0277407 A1
(43) Pub. Date: Aug. 22, 2024

(54) MICROWAVE CATHETERS FOR HIGH-POWER THERMAL ABLATION

(71) Applicants: Kansas State University Research Foundation, Manhattan, KS (US); phenoMapper, LLC, San Jose, CA (US)

(72) Inventors: Jan Sebek, Manhattan, KS (US); Punit Prakash, Manhattan, KS (US); Henky Wibowo, San Jose, CA (US)

(21) Appl. No.: 18/044,012

(22) PCT Filed: Sep. 3, 2021
(86) PCT No.: PCT/US2021/049119

§ 371 Date: Mar. 3, 2023

Related U.S. Application Data
(60) Provisional application No. 63/074,165, filed on Sep. 3, 2020.

(51) Int. Cl. A61B 18/18; A61B 18/00; A61B 34/20; A61B 90/00

(52) U.S. Cl. CPC A61B 18/1815; A61B 34/20; A61B 90/37; A61B 90/39; A61B 2018/00023; A61B 2018/00083; A61B 2018/00178; A61B 2018/00577; A61B 2018/00702; A61B 2018/00791; A61B 2018/00886; A61B 2018/1823; A61B 2018/1838; A61B 2018/1846; A61B 2018/1853; A61B 2018/1861; A61B 2090/376; A61B 2090/3966

(57) ABSTRACT

Catheter-based applicators incorporating a microwave ablation antenna are provided. The applicator can be flexible and suitable for delivering ablation to target tissues via endoscopic approaches as well as intracavitary/intraluminal approaches. The applicators are configured to create large volume thermal ablation zones with minimal radiation losses along the applicator axis traveling proximal to the ablation zone. This effect is accomplished by employing a backward-facing choke element and/or a multi-slotted antenna having a plurality of spaced-apart conducting elements encircling the antenna.

### CROSS-REFERENCE TO RELATED APPLICATIONS

This application claims the benefit of U.S. Provisional Application Ser. No. 63/074,165, filed Sep. 3, 2020, entitled MICROWAVE CATHETERS FOR HIGH-POWER THERMAL ABLATION, which is hereby incorporated in its entirety by reference herein.

### STATEMENT REGARDING FEDERALLY SPONSORED RESEARCH

This invention was made with government support under Contract No. R01 CA 218357 awarded by the National Institutes of Health. The government has certain rights in the invention

### BACKGROUND OF THE INVENTION

### Field of the Invention

The present invention is generally directed toward tubular-based applicators incorporating a microwave antenna for minimally invasive interventional application. In certain embodiments, the applicator is flexible and suitable for creating large volume thermal ablation zones in a range of tissues, including lung tissue, blood vessels, and other surrounding anatomy. Other possible embodiments include hyperthermia treatment and combination therapy with localized drug therapy and radiation therapy. The applicator is suitable for delivering thermal energy to targets via endoscopic approach or delivery device such as extended working channel, guide sheath, and/or robotic arm, as well as via intracavitary/intraluminal approaches.

### Description of the Prior Art

Microwave ablation (MWA) is an increasingly used thermal therapy modality for minimally-invasive treatment of tumors and benign disease. These procedures may be performed minimally invasively (typically under guidance of ultrasound, or computerized tomography guidance), laparoscopically, or under open surgery. MWA has found clinical applications in the treatment of tumors in the liver, kidney, lung, and bone, as well for treatment of cardiac arrhythmias, ablation of benign prostate tissue to treat hyperplasia (BPH), ablation of the uterine endometrial lining to treat menorrhagia, ablation of the esophageal wall for treating Barrett's esophagus and GERD, ablation of nerves for treating back pain, and ablation of renal nerves for treating chronic high blood pressure.

During an ablation procedure, an antenna is inserted into the target tissue or placed in close proximity thereto and radiates electromagnetic power at microwave frequencies; most currently available devices operate within frequency bands approved for industrial, scientific, and medical (ISM) use, centered at 915 MHZ, 2.45 GHZ, or higher frequencies. Electromagnetic power radiated from the antenna is deposited in the electromagnetic lossy tissue leading to heating via dielectric hysteresis. While thermal damage following ablation is a complex function of the time-temperature history during heating, temperatures in excess of 60° C. lead to near-instantaneous cell death by coagulative necrosis. Irreversible, but not lethal, thermal damage may occur in cells heated above 42° C. A fundamental principal of successful ablation is the creation of an ablation zone that sufficiently covers the entire target while providing a margin of safety for adjacent tissues. Thus, generation of a spherical ablation zone can be advantageous in order to better control the treatment being provided.

In some conventional microwave ablation instruments, the antenna can be connected to a feed cable, which feeds current to the antenna. However, a back current may be reflected along the feed cable away from the antenna. This back current decreases the power delivered to the ablation zone around the antenna in the targeted tissue and can create an undesirable tear-shaped ablation zone. A choke can be used to mitigate these back currents running on the outer conductor of a coaxial feed cable that supplies electromagnetic energy to the antenna. WO 2019/029906 illustrates several types of chokes used in the construction of microwave ablation probes that are arranged to reduce power reflected from the applicator. For example, the '906 publication illustrates a “forward-facing” choke in which the electrical connection between the choke and outer conductor does not occur at the distal end of the outer conductor, but rather the connection is located in a more proximal location along the length of the feed cable. This choke forms a pocket whose distal end (facing the antenna) is open. The '906 publication also describes several spiral-shaped choke configurations. But, again, even in these embodiments, the connection of the choke to the outer conductor is spaced from the outer conductor's distal end.

Other examples of microwave ablation instruments are described in US 22014/0276739, M. Cavagnaro et al., IEEE Trans Biomed Eng, 58(4): 949-59, 2010, and R. Nevels et al., IEEE Trans Biomed Eng, 45(7): 885-90, 2010.

### SUMMARY OF THE INVENTION

According to one embodiment of the present invention there is provided an electrosurgical device for tissue ablation. The device comprises an elongate catheter, a transmission cable at least partially located within the catheter, an antenna extending from the distal end of the cable and configured to emit microwave energy therefrom, and an ablation shape enhancer element that surrounds a least a portion of the transmission cable. Embodiments of the shape enhancer elements include a choke or a sleeve element. The catheter comprises a distal end that is configured for insertion into a body comprising the target tissue for ablation. The transmission cable comprises a cable proximal end configured to be connected to a power source for generating microwave power and a cable distal end. The transmission cable comprises at least one inner conductor and an outer conductor that are electrically isolated from each other by a dielectric material. The choke element comprises choke distal and proximal ends with the choke element distal end being electrically connected to the outer conductor. The choke element proximal end is radially spaced from the outer conductor.

According to another embodiment of the present invention, there is provided an electrosurgical device for tissue ablation. Like the previous embodiment, this embodiment also comprises an elongate catheter having proximal and distal catheter ends with the catheter distal end being configured for insertion into a body comprising the tissue targeted for ablation. A transmission cable is at least partially located within the catheter and comprises a cable proximal end configured to be connected to a power source for generating microwave power and a cable distal end. The transmission cable comprises at least one inner conductor and an outer conductor that are electrically isolated from each other by a dielectric material. The device further comprises an antenna extending from the cable distal end and configured to emit microwave power therefrom. The device further comprises a plurality of spaced-apart, annular conductive elements positioned around the antenna. At least one of the annular conductive elements is electrically connected to the inner conductor and at least one other of the annular conductive elements is electrically isolated from the inner conductor.

According to still another embodiment of the present invention, there is provided a method of ablating tissue within a body. The method comprises inserting at least one electrosurgical device as described herein into the body containing the tissue to be ablated. The device antenna is then positioned into or adjacent to the tissue to be ablated. The device is then activated thereby causing the antenna to emit electromagnetic power that is sufficiently strong to cause ablation of the tissue.

According to still another embodiment of the present invention, there is provided a method of ablating tissue within a body. The method comprises positioning the device antenna into or adjacent to the tissue to be ablated; activating the device thereby causing the antenna to emit electromagnetic power that is sufficiently strong to cause ablation of the tissue and define a predicted ablation zone; and changing a shape of the predicted ablation zone by circulating a flow of cooling water along the device antenna.

### BRIEF DESCRIPTION OF THE DRAWINGS

FIG. 1 is a schematic illustration of a microwave applicator according to one embodiment of the present invention;

FIG. 1A is a cross section of the microwave applicator taken along line 1A-1A in FIG. 1;

FIG. 2 shows simulated electromagnetic heat losses of the microwave applicator at different power levels and times during an ablation treatment;

FIG. 3 shows the results of coupled electromagnetic-thermal simulations in which the shape of the ablation zone at different power settings and ablation times is represented by means of a 60° C. isotherm contour;

FIG. 4 contains photographs of ex vivo testing of the microwave applicator at two power/time combinations;

FIG. 5 is a schematic illustration of another embodiment of a microwave applicator according to the present invention;

FIG. 6 shows simulated electromagnetic heat losses of the microwave applicator at different power levels and times during an ablation treatment; in

FIG. 7 shows the results of coupled electromagnetic-thermal simulations in which the shape of the ablation zone at different power settings and ablation times is represented by means of a 60° C. isotherm contour;

FIG. 8 contains photographs of ex vivo testing of the microwave applicator at two power/time combinations;

FIG. 9 shows simulated electromagnetic heat losses of the microwave applicator at different power levels and times during an ablation treatment;

FIG. 10 is a schematic illustration of another embodiment of a microwave applicator according to the present invention;

FIG. 10A is a cross section of the microwave applicator taken along line 10A-10A in FIG. 10;

FIG. 10B is a cross section of the microwave applicator taken along line 10B-10B in FIG. 10;

FIG. 11 is an elevation view of a conductive sheet of the microwave applicator depicted in FIG. 10, showing the conductive sheet in a flat configuration, with the sheet including a series of slitted openings;

FIG. 12 is a fragmentary elevation view of the microwave applicator depicted in FIG. 10, showing the conductive sheet wrapped around a transmission cable of the microwave applicator to form a choke element;

FIG. 13 is a fragmentary schematic illustration of another embodiment of a microwave applicator according to the present invention, depicting a choke element that includes a braided tube; and

FIG. 14 is a fragmentary schematic illustration of another embodiment of a microwave applicator according to the present invention, depicting a choke element that includes a coiled wire.

While the drawings do not necessarily provide exact dimensions or tolerances for the illustrated components or structures, the drawings are to scale with respect to the relationships between the components of the structures illustrated in the drawings.

### DETAILED DESCRIPTION OF THE PREFERRED EMBODIMENT

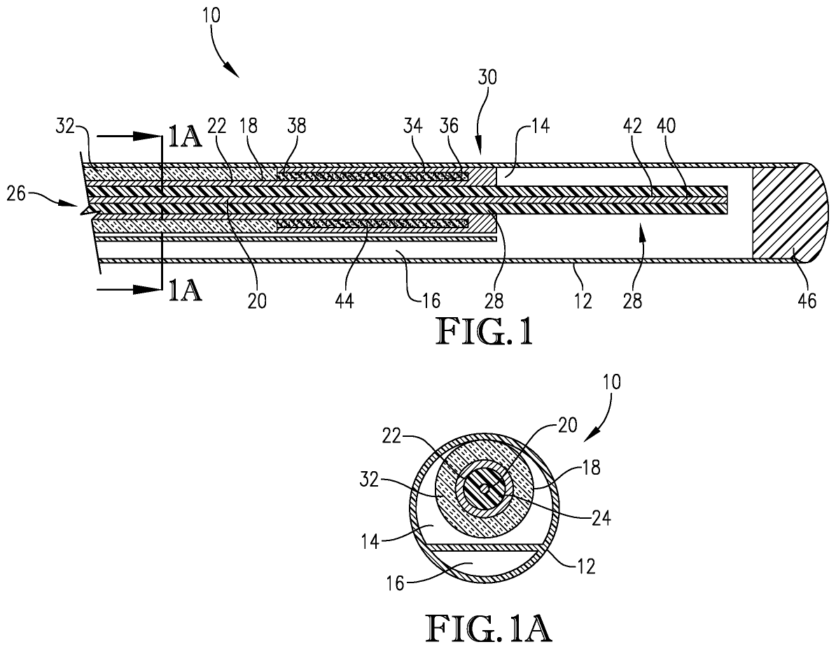

Creating large volume microwave ablation zones via an endoscopic approach requires applicators that: (1) are small enough to fit within the instrument channel of the endoscope (i.e. <2 mm O.D.), and ˜1 m long to access sites in diverse anatomic locations; (2) incorporate strategies for managing waste-heating due to microwave attenuation within flexible cables; (3) deliver sufficient power to targeted tissue to generate ablation zones of adequate size; (4) yield ablation zones of desired axial ratio; and (5) maintain structural integrity during high power ablation. Turning to FIG. 1, a microwave ablation device 10 according to one embodiment of the present invention is depicted. The microwave ablation device 10 enables creation of large volume (e.g., 1-5 cm diameter) ablation zones that are near-spherical in shape. As described below, the device 10 is preferably configured to include a flexible catheter to facilitate ablation of targets via an endoscopic approach (as used herein, an endoscope generally refers to and includes endoscopes, bronchoscopes, cystoscopes, hysteroscopes, and other similar instruments). However, the principles of the present invention also apply to rigid devices suitable for direct insertion into targets via a percutaneous image-guided approach, or via a surgical approach.

Device 10 comprises an elongate catheter 12 that is preferably made from a lossless or low-loss dielectric material, such as a synthetic resin material (e.g., elastomeric or plastic materials such as polyimide, PTFE, fiberglass, and polyether ketone (PEEK) tubing), a dielectric material, or a ceramic material. In this regard, the catheter 12, and thus device 10, is preferably flexible. It is within the scope of the present invention for catheter 12 to be formed from an electrically conductive material, such as a metal (e.g., stainless steel), and thus produce a rigid device suitable for insertion without an endoscope. However, in certain embodiments, the use of an elastomeric or plastic catheter material reduces adhesion of ablated tissue to the device 10 during use thereby facilitating easier insertion and withdrawal of the device into and from the patient's body. In alternative configurations, catheter 12 may be formed from conjoined sections of plastic, fiberglass, metal, or other materials to achieve the desired durability or rigidity. Preferably, the overall outer diameter of the device is 0.083″ (14 gauge), but the concepts described herein could be adapted for devices as small as 0.053″ (17 gauge). It is understood that device 10 may have any diameter that is suitable for a given application including outer diameters greater than 14 gauge or smaller than 17 gauge.

In certain embodiments, catheter 12 comprises a multi-lumen catheter. As can be seen in the cross-section (taken along line C-C) of FIG. 1, catheter 12 comprises a primary lumen 14 and a secondary lumen 16. A transmission cable 18 is at least partially located within lumen 14. Secondary lumen 16 can be used as a part of a closed-loop flow circuit to circulate a cooling fluid through device 10 to remove waste heat along the device's shaft thereby helping to maintain the structural integrity of the transmission cable 18. The coolant flow also helps to limit heating of tissue adjacent to the catheter 12, thereby minimizing heating of non-target tissue as well as limiting desiccation of tissue within the ablation zone. In certain embodiments, water is a preferred cooling fluid for circulation within device 10; however, other cooling fluids may be used such as saline, FLUORINERT, liquid chlorodifluoromethane, nitrous oxide, nitrogen, carbon dioxide and air. Besides the catheter configuration depicted in FIG. 1, other catheter structures, such as concentric flexible catheters may also be used.

In one or more embodiments, transmission cable 18 comprises a coaxial cable having an inner conductor 20, an outer conductor 22, and a dielectric material 24 disposed therebetween. Although, it is within the scope of the invention for other two-wire assemblies to be used besides a coaxial cable. In certain embodiments, the inner conductor 20 comprises, for example, copper, silver, gold, silver-plated copper weld, or any combination thereof, and the outer conductor 22 comprises a conductive metal, for example, copper or steel. The coaxial cable 18 may be constructed from either solid (semi-rigid) or braided inner and outer conductors. In the case of a flexible device 10, outer conductor 22 may be a woven metallic (e.g., copper) shield. Preferably, the conductors are made from non-magnetic materials which may facilitate use of device 10 in an MRI scanner. Otherwise, alternate materials, such as stainless steel, may be used which may impart added stiffness to device 10 thereby enhancing rigidity. The dielectric material 24 may comprise, for example, polytetrafluoroethylene, air, polyethylene, alumina, nylon, and combinations thereof.

The proximal end 26 of the transmission cable 18 is configured to be coupled with an electromagnetic signal generator (not shown). The proximal end 26 may comprise an SMA connector or other structure (e.g., N-type, BNC, QMA, or other connectors) that is suitable for connecting the transmission cable 18 to the signal generator. An antenna 28 extends from the distal end 30 of the transmission cable 18 that is configured to emit microwave power therefrom that is sufficiently strong to cause tissue ablation. As illustrated, antenna 28 is a monopole antenna, but it is within the scope of the present invention for other types of antennae to be used including slot, dipole, and helical radiating antennae.

The embodiment of the antenna 28 depicted in FIG. 1 comprises a portion of transmission cable 18 in which a portion of the outer jacket 32 and outer conductor 22 have been removed thereby exposing a portion of the dielectric material 24 which surrounds a segment of the inner conductor 20. Generally, the antenna radiating element 40 is covered by an insulating material 42, such as polytetrafluoroethylene, which can be the same or different from the dielectric material 24. In certain embodiments, the insulating material 42 has low electrical conductivity and a high temperature rating. The presence of the insulating material 42 prevents direct contact between the radiating element 40 and the walls of catheter 12, thereby minimizing the likelihood of leaks, and serving to center/anchor the antenna 28 within the catheter 12.

The device 10 further comprises a choke element 34 that surrounds at least a portion of the transmission cable 18. The choke element 34 is configured to limit backward currents along the transmission cable 18. The choke element 34 comprises distal 36 and proximal 38 ends. The choke element distal end 36 is electrically connected to the outer conductor 22, whereas the proximal end 38 is radially spaced from outer conductor 22. By connecting the choke element 34 at the feed point of the antenna 28, backward radiation at the proximal end 38 of the choke is cut off. Unlike conventional chokes that are open at the distal end (i.e., face forward toward the antenna), choke element 34 faces backwards toward the catheter proximal end 26.

The choke element 34 preferably serves as an ablation shape enhancer. However, as will be explained below, the ablation device may include an alternative ablation shape enhancing element, such as an alternative choke element.

In one or more embodiments, the choke element 34 comprises thin wall electrically conducting sheets. In other embodiments, the choke element 34 may be constructed from a segment of the outer conductor 22 that is peeled back toward the proximal end 26. The choke element 34 may comprise any electrically conducting material, such as those from which inner conductor 20 and outer conductor 22 are formed. However, the choke element 34 need not comprise the same conducting material as the inner and outer conductors. In one or more embodiments, the choke element 34 is electrically isolated from the transmission cable 18 (except for the point of electrical connection between the choke element and the outer conductor 22), and in particular from the outer conductor 22, using an insulative material 44, such as any of those described herein, including silicone. In preferred embodiments, the insulative material is flexible and does not alter the device's flexibility and mechanical properties. Moreover, in certain embodiments, the insulative material 44 helps maintain the position of the choke element 34 at a suitable distance from the transmission cable 18.

In certain embodiments, but not necessarily all embodiments, it can be desirable for some degree of symmetry to exist between the choke element 34 and the antenna 28. In one aspect, the length of the choke element 34 is very similar to the length of the antenna 28. In one or more embodiments, the ratio of the choke element length to the antenna length is from about 0.75:1 to about 1.5:1, from about 0.8:1 to about 1.25:1, from about 0.9:1 to about 1.1:1, or about 1:1. In certain embodiments, the choke element 34 is formed from a material having a conductivity of at least 10⁴ S/m, at least 10⁵ S/m, at least 10⁶ S/m, or at least 10⁷ S/m at 20° C. Preferred materials for the choke element 34 include copper, gold, silver, and stainless steel.

In one or more embodiments, the choke element 34 is configured so that it does not appreciably alter the flexibility of the catheter. In particular embodiments, the bend radius for device 10 is 1 cm or less, preferably 7.5 mm or less, or more preferably 5 mm or less.

Device 10 may also comprise a tip 46 that seals the distal end of the catheter 12. In certain embodiments, the tip 46 may comprise an epoxy or other suitable material. However, tip 46 may also be a trocar tip to guide insertion of the device 10 into tissues.

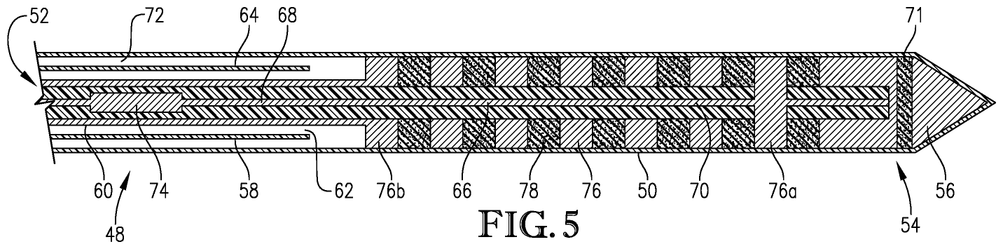

Another embodiment of a microwave ablation device 48 is shown in FIG. 5. Like device 10 of FIG. 1, device 48 comprises an elongate catheter 50 having proximal 52 and distal 54 catheter ends. In the illustrated embodiment, the catheter distal end 54 includes a trocar tip 56 to assist with insertion of the catheter into the body. However, as described above, other types of tips may be used without departing from the scope of the invention.

Device 48 comprises a tubular member 58 that is carried inside catheter 50, and preferably concentric with catheter 50, although this need not always be the case. A transmission cable 60 is at least partially located within the catheter 50, and preferably within the lumen 62 created by tubular member 58. The proximal end of the cable is configured to be connected to a power source (not shown) for generating microwave power. The transmission cable 60 is preferably a coaxial cable that comprises an outer conductor 64, and inner conductor 66, and a dielectric material 68 located therebetween that isolates the conductors from each other. The conductors 64, 66 and dielectric material 68 may be formed from the same materials as described above for device 10. An antenna 70 extends from the distal end of the transmission cable 60 and is configured to emit microwave power therefrom. The antenna 70 is depicted in FIG. 5 as a monopole antenna, but any type of antenna previously described herein can be used. As illustrated, the antenna 70 comprises an extension of the inner conductor 66 surrounded by the insulative, dielectric material 68. The distal end of the antenna 70, including dielectric material 68 can be secured or anchored to the rigid tip 56 to maintain the position of the antenna within the device 48. Moreover, a segment of silicone (or other electric insulator) 71 can be provided as a seal between the catheter distal end 54 and tip 56.

The catheter 50 and tubular member 58 can also cooperate to define an annular region 72 which, along with lumen 62, can be used to circulate a cooling fluid through device 10, such as any of the cooling fluids previously described herein.

The transmission cable 60 can also be provided with a matching network element 74, which is preferably electrically coupled to inner conductor 66. The matching network helps to match the antenna's impedance to the feeding transmission line, and thus minimizing reflected power along the feeding line.

The device 48 further comprises a plurality of spaced-apart, annular conductive elements 76 positioned around the antenna 70. In one or more embodiments, the annular conductive elements 76 comprise coils, which may be fabricated from a fine wire, that are wound around the dielectric material 68. In alternate embodiments, the annular conductive elements 76 may comprise discs, having a central orifice through which the inner conductor 66 and/or dielectric material 68 may pass. The annular conductive elements 76 may comprise any of the same conductive materials from which inner conductor 66 and outer conductor 64 may be made, as described above, or from other electrically conductive materials.

At least one, and preferably only one, of the annular conductive elements 76 is electrically connected to the inner conductor 66. At least one, and preferably all other, annular conductive elements 76 are electrically isolated from the inner conductor 76. In one or more embodiments, the annular conductive element 76 that is electrically connected to the inner conductor 66 comprises the most distal conductive element 76a of the series of elements. In one or more embodiments, at least one annular conductive element 76b, and preferably only one of the annular conductive elements, is electrically connected to the outer conductor. In preferred embodiments, conductive element 76b comprises the most proximal of the series of conductive elements.

In one or more embodiments, the annular conductive elements 76 are separated laterally from each other by an insulating material 78, such as silicone, or any other insulating material described herein including the same material as the dielectric material 68. Preferably, the insulating material comprises a material that is stable at temperatures of about 150° C. or higher. The insulating material 78 not only isolates the conductive elements 76 but helps to maintain their spatial separation. In one or more embodiments, the spacing between the conductive elements 76 is regular and uniform throughout the series. However, this need not always be the case. The number of conductive elements 76 encircling the antenna 70 may also vary depending upon factors such as the length of the antenna. In certain embodiments, the number of conductive elements can range from 3 to 15, 4 to 12, or 5 to 10. As depicted in FIG. 5, device 48 comprises seven conductive elements.

As can be seen in FIG. 5, not only is the spacing between conductive elements 76 about the same, the width of each conductive element is approximately the same as the width of the gap (i.e., the width of the insulating material 78) between elements. Again, this need not always be the case, however. In one or more embodiments, the ratio of the width of the conductive elements 76 to the width of the insulating material 78 is from about 0.75:1 to about 1.5:1, from about 0.8:1 to about 1.25:1, from about 0.9:1 to about 1.1:1, or about 1:1.

In one or more embodiments, the device 48 may also include a choke element (not shown in FIG. 5), such as choke element 34 from FIG. 1, that surrounds at least a portion of the transmission cable 60. The choke element can be a backward facing choke element 34 as described above, or any other conventional choke known in the art.

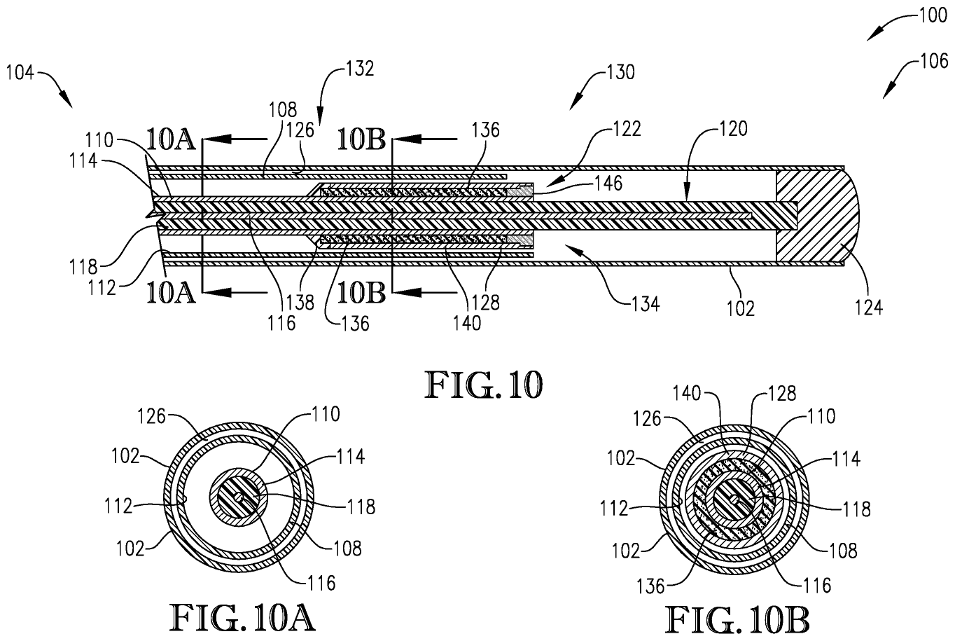

Another embodiment of a microwave ablation device 100 is shown in FIGS. 10-12. Similar to device 10 of FIG. 1, device 100 comprises an elongate catheter 102 having proximal 104 and distal 106 catheter ends. Device 100 comprises a tubular member 108 that is carried inside catheter 102 and is preferably concentric with catheter 102. Alternative device embodiments may include a tubular member that is alternatively constructed and/or positioned relative to the catheter.

A transmission cable 110 is at least partially located within the catheter 102, and preferably within a lumen 112 defined by tubular member 108. The proximal end of the cable is configured to be connected to a power source (not shown) for generating microwave power. The transmission cable 110 is preferably a coaxial cable that comprises an outer conductor 114, an inner conductor 116, and a dielectric material 118 located therebetween that isolates the conductors from each other. The conductors 114, 116 and dielectric material 118 may be formed from the same materials as described above for device 10.

The transmission cable 110 preferably comprises a flexible transmission line. Although the transmission cable 110 in FIG. 10 comprises a coaxial cable transmission line, alternative device embodiments may include an alternative transmission line such as a two-wire line.

An antenna 120 extends from the distal end of the transmission cable 110 and is configured to emit microwave power therefrom. The antenna 120 is depicted in FIG. 10 as a monopole antenna, but any type of antenna previously described herein can be used. As illustrated, the antenna 120 comprises an extension of the inner conductor 116 surrounded by the insulative, dielectric material 118.

The antenna 120 preferably comprises a monopole antenna, where the outer conductor has been stripped off along the length of the antenna 120. It is preferable to leave the dielectric layer 118 intact along the antenna length. However, in alternative embodiments, a bare wire antenna may be constructed with the dielectric removed. The resonant antenna length may be variously sized depending on the materials surrounding the exposed inner conductor. The proximal end of the antenna 120 (i.e., the point at which the outer conductor is first removed to expose the inner conductor) comprises an antenna junction 122.

The distal end of the dielectric material 118 is preferably embedded within a distal tip 124, which seals the distal end of the catheter 102. In certain embodiments, the tip 124 may comprise an epoxy or other suitable material. However, tip 124 may also be a trocar tip to guide insertion of the device 100 into tissues. The antenna 120 is preferably terminated at a distal end that is spaced proximally relative to the distal end of the dielectric material 118 and relative to the epoxy tip 122. That is, the coaxial cable dielectric material 118 is extended past the inner conductor 116. This allows for bonding the extended portion of the dielectric material 118 within the catheter tip. This can be achieved by using an adhesive plug (e.g., epoxy) to form a sealed plug/tip for the catheter, and to approximately center the antenna within the catheter. Preferably, the catheter tubing material is a material that can tolerate high temperatures (in excess of 150° C.). Examples of such materials include PTFE, Polyimide, or blends of polyimide/PTFE. The material should also be selected to provide good “pushability” of the device. That is, longitudinal forces applied at the proximal end of the catheter should be translated to the distal tip of the catheter enabling the device to be pushed through tissue or lumens without kinking. As described below, embodiments of the distal tip may include one or more materials (e.g., a ceramic material, a metallic material, or combinations thereof) embedded within the tip to facilitate visualization of the distal tip via fluoroscopy.

The catheter 102 and tubular member 108 cooperatively define an annular lumen 126 which, along with lumen 112, may be used to circulate a cooling fluid through device 100, such as any of the cooling fluids previously described herein. Similar to the device 10, the device 100 comprises a dual-lumen catheter that permits closed-circuit circulation of water. In the depicted embodiment, lumen 112 receives the transmission cable 110 and provides a flow path for the cooling fluid. The other lumen 126 then provides a return path for the cooling fluid.

The device 100 further comprises a choke element 128 that surrounds at least a portion of the transmission cable 110. Similar to the choke of device 10, the choke element 128 is configured to limit backward currents along the transmission cable 110. Choke element 128 presents distal 130 and proximal 132 ends. The choke element distal end 130 is electrically connected to the outer conductor 114, whereas the proximal end 132 is radially spaced from outer conductor 114. As with device 10, by connecting the choke element 128 only at a feed point 134 of the antenna 120, backward radiation at the proximal end 132 of the choke is cut off.

Choke element 128 is electrically isolated from the transmission cable 110 (except for the point of electrical connection between the choke element 128 and the outer conductor 114), and in particular from the outer conductor 114, using an insulative layer 136, such as any of those described herein, including silicone. Choke element 128 is further electrically isolated from the transmission cable 110 using a proximal end seal 138.

In preferred embodiments, the insulative layer 136 comprises a flexible material and does not alter the device's flexibility and mechanical properties. Moreover, in certain embodiments, the insulative layer 136 helps maintain the position of the choke element 128 at a suitable distance from the transmission cable 110.

Insulative layer 136 may include a silicone material (with a relative permittivity of about 3.5-4). It has been found that the use of silicone in the insulative layer is desirable because the silicone material is relatively soft and flexible and does not restrict the flexibility of the catheter. The material on the outer surface of the choke element 126 preferably comprises water (which has a relative permittivity of about 78 at room temperature). Water is selected as it performs two roles: it can be circulated through the applicator to remove waste heat from the coaxial cable. Further, it provides a high permittivity material to constrain the suitable length of the third conductor. Although the use of water is preferred as a material on the outer surface of the choke element, the outer surface of the choke element may be provided with one or more alternative materials (such as an alternative material with high permittivity).

The end seal 138 preferably comprises an epoxy seal and located at the proximal choke end 132 to restrict water from seeping into the choke element 128.

Choke functionality is preferably a function of the length and thickness of the insulative layer 136 between the outer conductor 114 and the choke element 128; and the length and thickness of the material on the outer surface of the choke element 128. Again, the material on the outer surface of the choke element 128 preferably comprises water.

To restrict radiation to the catheter distal end, the choke must be properly designed to limit backward radiation. For effective operation, the choke design may be adjusted based on the: length of the insulative layer 136, thickness of the insulating layer 136, length of the choke element 128, relative permittivity of the insulative layer 136, and the relative permittivity of the material on the outer surface of the choke element 128. Generally, the use of a relatively thick layer of materials between the outer conductor 114 and the choke element 128, as well as a relatively thick layer of material on the outer surface of the choke element 128, permits the use of a relatively shorter choke element 128. Furthermore, materials with higher permittivity permit the use of a relatively shorter choke element 128.

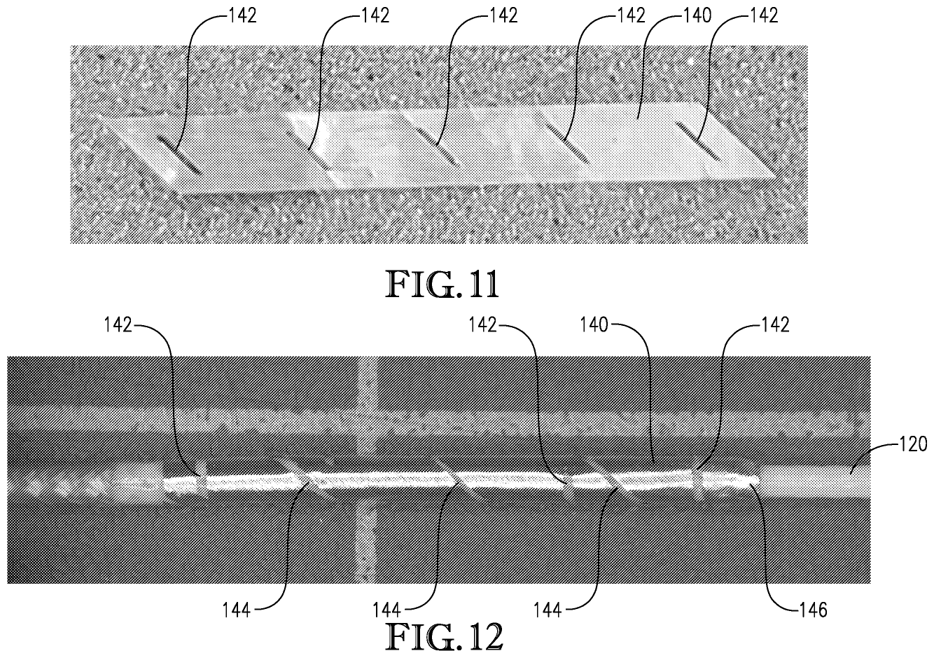

Turning to FIGS. 11 and 12, in one or more embodiments, the choke element 128 comprises one or more thin-wall electrically conducting sheets. Preferably, the choke element 128 is formed by wrapping one or more conducting sheets 140 in a helical direction along the outer conductor 114 so that the choke element has a tubular shape. The depicted sheets 140 include a plurality of slitted openings 142 spaced along the length of the sheets 140 (see FIGS. 11 and 12). When wrapped around the transmission cable 110, the sheets 140 also define a helical slot 144 that extends between distal 130 and proximal 132 choke ends (see FIG. 12).

To maintain flexibility of the overall choke structure, it is preferable to form the choke element 128 with gaps for providing stress relief as the catheter bends. One method for achieving this is to wrap one or more sheets of conducting material (e.g., copper film) around the insulative layer 136 and seal the sheet(s) to form a roll. Openings may then be formed in the sheet(s) (e.g., by making cuts with a scalpel or a laser cutter) to introduce gaps that provide stress relief. In embodiments within the scope of the present invention, the pattern of openings may be horizontal, vertical, diagonal, and/or of other patterns. Although the sheets 140 preferably include a series of elongated slitted openings 142, embodiments of the choke element may include other types of perforations, such as a distributed array of circular holes.

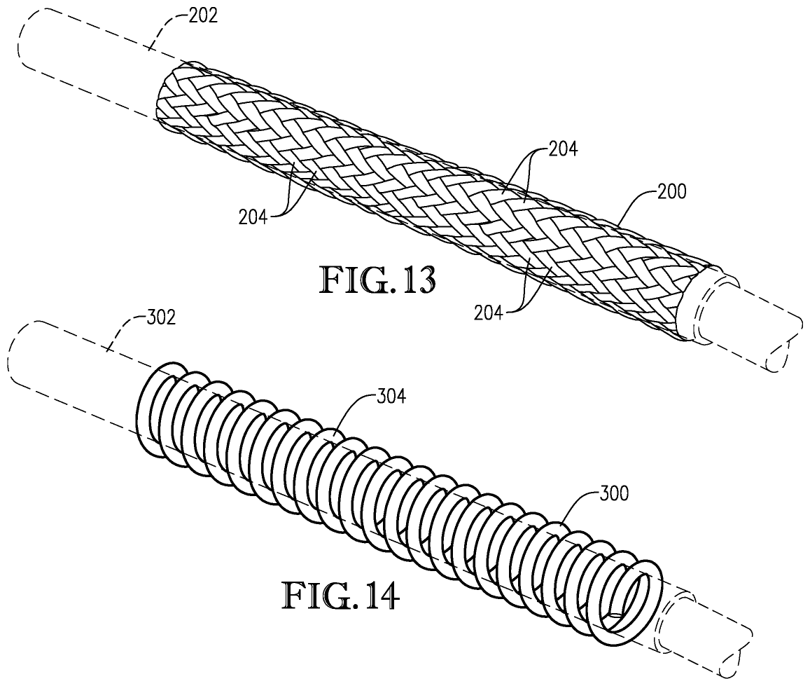

In alternative embodiments within the scope of at least certain aspects of the present invention, an alternative conductor may be wrapped around the insulative layer. An alternative choke element may be formed of a material element other than a continuous sheet that forms a tubular-shaped element with one or more openings. For instance, alternative embodiments of the conductive layer may be formed from a mesh, a stent, a spring, braided cable, or other material. As shown in FIG. 13, alternative choke element 200 may be secured on transmission cable 202 and include a braided tube with multiple wire braids 204. As shown in FIG. 14, an alternative choke element 300 may be secured on transmission cable 302 and include a wire coil 304, which is shaped like a coil spring. Preferably, such alternative embodiments are electrically coupled to the coaxial cable outer conductor at the distal end of the choke element. Alternative embodiments of the choke element preferably surround the insulative layer and are preferably surrounded by a higher permittivity material (e.g., water). For at least certain aspects of the present invention, embodiments of the choke element may include a continuous tube element that extends endless about the transmission cable, such that the tube element is not wrapped around the transmission cable.

Although the disclosed choke elements are preferred for providing the ablation device with an ablation shape enhancer, other embodiments of choke elements are within the scope of the present invention. Furthermore, ablation device embodiments within the ambit of at least certain aspects of the present invention may include one or more other elements that serve as an ablation shape enhancer.

Turning to FIGS. 10-12, the choke element 128 may comprise any electrically conducting material, such as those from which inner conductor 116 and outer conductor 114 are formed. However, the choke element 128 need not comprise the same conducting material as the inner and outer conductors. The illustrated choke element 128 is electrically isolated from the transmission cable 110 except for a solder or conductive-based material joint 146 between the choke element 128 and the outer conductor 114.

For the present invention, the distal end of choke element 128 is electrically coupled to the outer conductor 114 so that the choke element 126 is shorted at the antenna junction 122. In the depicted embodiment, solder joint 146 includes conductive-based joint material that fixes and electrically connects the choke element 126 and the outer conductor 114 to one another. This configuration ensures that reverse flowing currents are suppressed at the source. This configuration also permits optimal sizing of the choke structure through asymmetric combination of materials with permittivity on either side of the choke element.

In certain embodiments, but not necessarily all embodiments, it can be desirable for some degree of symmetry to exist between the choke element 128 and the antenna 120. In one aspect, the length of the choke element 128 is very similar to the length of the antenna 120. In one or more embodiments, the ratio of the choke element length to the antenna length is from about 0.75:1 to about 1.5:1, from about 0.8:1 to about 1.25:1, from about 0.9:1 to about 1.1:1, or about 1:1. In certain embodiments, the choke element 128 is formed from a material having a conductivity of at least 10⁴ S/m, at least 10⁵ S/m, at least 10⁶ S/m, or at least 10⁷ S/m at 20° C. Preferred materials for the choke element 128 include copper, gold, silver, and stainless steel.

In one or more embodiments, the choke element 128 is configured so that it does not appreciably alter the flexibility of the catheter. In particular embodiments, the bend radius for device 10 is 1 cm or less, preferably 7.5 mm or less, or more preferably 5 mm or less.

Embodiments of the ablation device also preferably facilitate identification of applicator position during fluoroscopy (2D X-ray projection) imaging. The electrical coupling between the choke element 128 and coaxial cable outer conductor 114, achievable with soldering, welding, gluing or other process, leads to a readily visible point on the applicator on fluoroscopic imaging. This enables ready identification of a key point of the catheter during an imaging process. In the illustrated embodiment, the solder material of the solder joint 138 preferably provides a doping element that serves as a radiopaque marker for identifying the location of the solder joint during an imaging process. Although the doping element preferably comprises a metal solder material, the doping element may include one or more alternative materials for applicator imaging. For instance, other materials with X-ray contrast, such as a ceramic material or a high-density material, may provide a radiopaque marker. It will also be understood that the joint between the choke element and the outer conductor may include an electrically-conductive epoxy or another electrically-conductive adhesive to electrically couple the choke element and the outer conductor.

It is also within the ambit of certain aspects of the present invention for one or more additional doping elements to be provided at one or more additional locations along the antenna. In alternative embodiments, an ablation device may have a doping element provided as part of a distal-most tip or trocar. For instance, the distal tip 124 may be configured to receive a solder element or ceramic element therein. With doping elements located at the joint 146 and the tip 124, the doping elements are correspondingly positioned at opposite ends of the antenna 120. Thus, the doping elements cooperatively “bracket” opposite ends of the antenna 120 so that the antenna ends may be “illuminated” or otherwise viewed during an imaging process (such as a fluoroscopy imaging process). Consequently, the location of the ablation device, particularly the antenna, may be monitored during the imaging process as the ablation device is placed relative to the target tissue.

It will be appreciated that the position of the ablation applicator relative to the targeted tumor is important for ensuring adequate thermal ablation of the target. Thus, during an ablation procedure, it is important to identify the position of the applicator relative to the target tumor. During bronchoscopy-guided ablation procedures, fluoroscopy imaging is widely used to visualize the position of the bronchoscope and additional interventional tools (e.g., the ablation applicator). Image-guided navigation systems further overlay the targeted tissue on the fluoroscopy image. The antenna junction is readily visible on fluoroscopy, due to the geometry and selection of materials in the applicator. Thus, the operator may utilize this position of the applicator, as inferred from the visualized junction position, to guide adjustment of the applicator position prior to starting the ablation procedure.

A goal of ablation procedures is to cover the entire tumor, and surrounding margin of normal tissue, with ablative heating. The extent of the margin depends on the targeted tissue and tumor. Depending on the applicator position within the tumor, the applied power level and ablation duration (collectively referred to as the energy dose), may vary in order to achieve an adequate treatment margin. This is due to the geometry of the applicator relative to the tumor, the tumor's geometry, the energy delivery pattern of the applicator, and the differences in the physical properties of the tumor tissue and surrounding normal tissue (such as the presence of other structures such as airways and blood vessels).

In practice, the physician operator may aim to guide the applicator to a desired position. However, due to the technical challenges associated with advancement of the bronchoscope and the applicator, and motion of the lungs and other structures, it is difficult to exactly achieve the desired target position. The actual achieved position may differ considerably from the desired position. Computational models may be instructive for identifying the selected energy dose. For a given tumor size/shape, computational models of ablation, which are developed prior to the ablation procedure, may identify the relationship between a candidate energy dose and ablation outcome (i.e., coverage of the tumor and minimum treatment margin) for a given applicator position. The models may then be used to update the estimated ablation outcome if the applicator position deviates from the studied position for a threshold distance (e.g., within a 5 mm sphere of the studied position). If all applicator positions within this sphere lead to acceptable estimated ablation outcome for the studied energy dose, this information can be indicated intra-procedurally by overlaying a target applicator position on the virtual fluoroscopy screen, in addition to the tumor boundary. Thus, the operator can aim to deliver the applicator to a position within the sphere.

Similarly, models can further be extended to indicate how the energy dose should be adapted to achieve an acceptable ablation outcome, if the applicator position deviates by a larger extent (for instance, if the positional deviation lies within 5-10 mm of the intended position. This information can then be conveyed to the physician operator intra-procedurally.

A key aspect to enable this approach is the identification of the applicator position on fluoroscopy. Materials that can be readily visualized on X-ray fluoroscopy provide a facile means to achieve this. Materials with density considerably different from background lung tissue, as well as considerably different density than plastic and water (materials within the applicator), can provide suitable contrast. The design of the choke element to be positioned immediately adjacent to the antenna junction enables this by providing ready visualization of a key point along the antenna axis. The location of key features along the applicators are preferably positioned along the distal-most end of the applicator, which is the active radiating length of the applicator. In particular, identifying the regions where the most intensive microwave power absorption is anticipated is preferable.

As noted previously, an electromagnetic power source is used to generate and transmit the desired microwave power to the applicator devices 10, 48, 100 described above. The electromagnetic power source may include a microwave signal generator, and optionally a DC power supply, a power amplifier, and a power monitor. In certain embodiments, the frequencies generated by the signal generator are similar to those that are associated with the frequencies typically used to heat water. In particularly preferred embodiments, the frequencies generated range from about 800 MHz to 6 GHz, from about 900 MHz to about 5 GHZ, or from about 1 GHz to about 3 GHZ, or about 915 MHz or about 2.45 GHz.

The operation of the devices 10, 48 and any peripheral accessories, such as sensors, may be monitored and controlled by a microprocessor, such as a personal computer or a handheld device. Alternatively, the operation of the devices 10, 48 and their peripheral accessories can be monitored and controlled by a user interface and control system that is integral with the electromagnetic power source.

The devices 10, 48, 100 described herein can be used in several applications for ablating tissue, such as a tumor, nerve, or other tissue, within the body of a human or animal. Generally, the device 10, 48, 100 is inserted into the body containing the tissue to be ablated. Insertion of the device may be carried out percutaneously, particularly when the device is equipped with a trocar tip 56 to create an opening in the skin. The device may also be used in open surgery or inserted laparoscopically, such as through an incision and/or through a trocar that has been previously inserted into the patient's body. In addition, in certain embodiments if the antenna is incorporated onto the distal end of a flexible cable assembly, the device may be used endoscopically or used within a body lumen (endo-luminally) directly such as within a vein or artery (endo-vascularly), or bronchoscopically. Once inside the patient's body, the device 10, 48, 100 and particularly the device antenna 28, 70, 120, is positioned adjacent or into to the tissue to be ablated, such as into an organ in which a tumor is located.

The use of choke elements 34, 128 and/or configuring the antenna 70 with a plurality of conductive elements 76, reduces the backflow of energy toward the proximal end of the transmission cable and helps to achieve an ablation zone within the tissue that has an axial ratio (the ratio of the dimensions of the ablation zone's major to minor axes) of from about 0.75:1 to about 1.3:1, from about 0.85:1 to about 1.2:1, from about 0.95:1 to about 1.1:1, or about 1:1.

### Example 1

A microwave application device was constructed according to the concepts depicted in FIG. 1 from a low-loss flexible coaxial cable with a 1.13 mm outer jacket diameter. A monopole radiator was created by stripping the jacket and outer conductor of the coaxial cable, leaving the inner conductor with dielectric behind. A choke element was positioned to limit feedline currents and electromagnetic radiation along the applicator axis traveling proximal to the ablation. The choke comprised Cu-PET tape or plate, which was wrapped around a thin high-temperature silicon layer and soldered at the junction point (point, where the outer conductor ends, and only dielectric and inner conductor continue) to the outer conductor. The length of the choke is a function of its dimensions (length and inner diameter) and the electrical properties of the material intervening between the choke and the coaxial cable. The optimal position of the choke is the antenna feed point (junction).

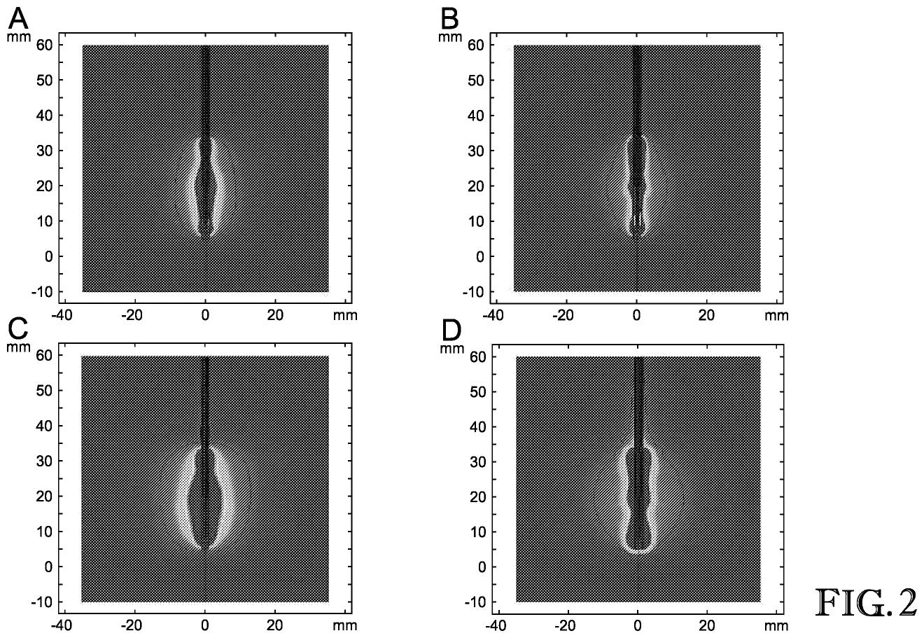

Simulations were performed to determine the electromagnetic heat losses and temperature profiles of the microwave applicator. FIG. 2 shows simulated electromagnetic heat losses of the microwave applicator at different power levels and times during an ablation treatment, namely electromagnetic heat loss of the applicator at 30 W and (A) 0s, (B) 10 min, and at 80 W and (C) 0s and (D) 10 min. These electromagnetic simulations reveal the main function of the choke, which is to eliminate or significantly suppress the propagation of E-field and therefore electromagnetic heat losses along the insertion track towards the applicator insertion point.

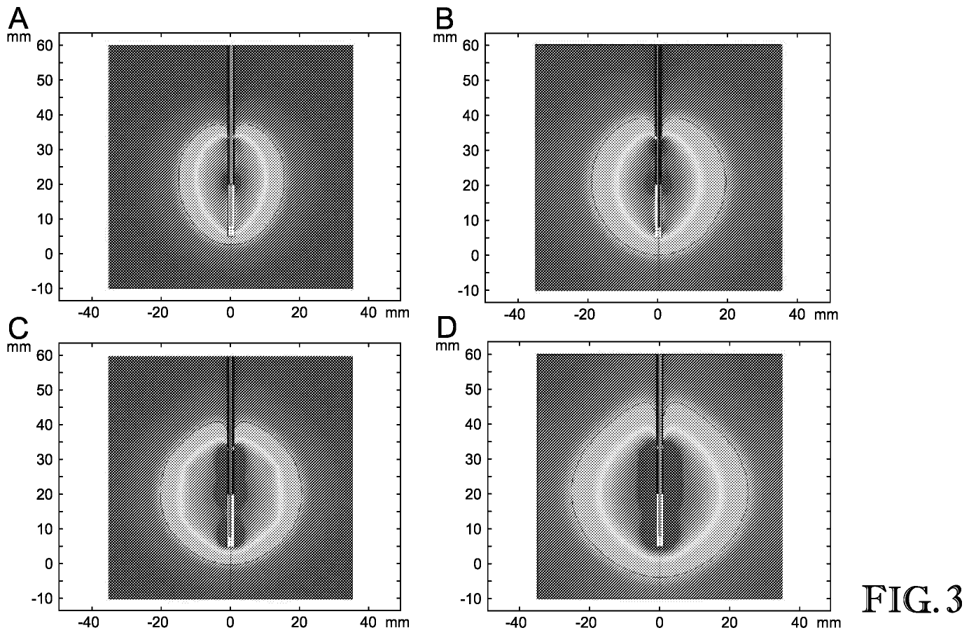

Ablation artifacts like skin burns, comet-tails or an oval shaped ablation zone are, however, very easily formed even if the radiation pattern does not yield any tail. This is due to the thermal conduction of the tissue and applicator along the antenna insertion track itself. Therefore, to verify the effect of the choke in combination with internal water cooling of the applicator shaft, coupled electromagnetic-thermal simulations were carried out to estimate the shape of ablation zone by means of 60° C. isotherm contour. FIG. 3 shows the result of these thermal simulations, namely temperature profiles of the microwave applicator at power 30 W and (A) 5 min, (B) 10 min, and at power 80 W and (C) 5 min and (D) 10 min. The black solid contour represents a 60° C. isotherm. Even at high power (80 W), the axial ratio (the ratio of major to minor axes) stays very close to 1 (˜0.95) confirming the efficiency of the developed ablation shape management strategy.

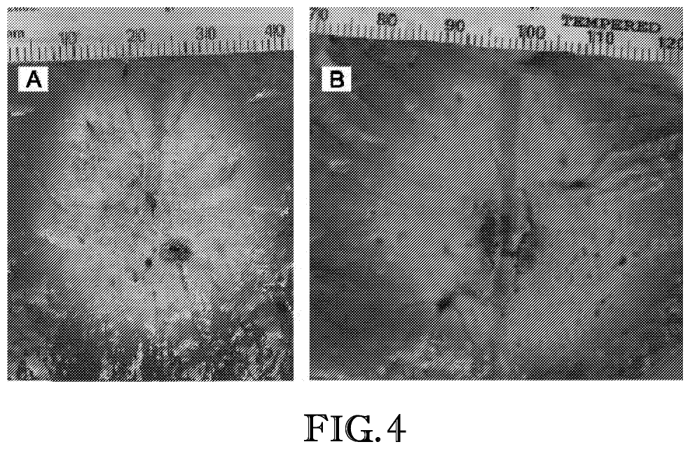

Results of the coupled electromagnetic-thermal simulations were verified experimentally on ex vivo liver tissue and at two power/time combinations. The resulting ablation zones are shown in FIG. 4 (ablation zones achieved in ex vivo liver tissue with (A) 23.85 W power applied for 10 min and (B) 31.8 W power applied for 10 min).

### Example 2

A microwave application device was constructed according to the concepts depicted in FIG. 5. UT47 semi-rigid coaxial cable was used. The multi-slot structure is created by stripping the outer conductor and pushing seven 1 mm-long coils around the inner conductor and dielectric. The last coil is shorted with the inner conductor, which provides improved impedance matching, when the tissue is desiccated. This applicator required a matching network, which can be implemented inside the coaxial cable. Water circulation is managed by two concentric polyimide tubes, where the outer one also serves as the applicator catheter. Finally, high-temperature silicone was utilized for holding the coils in their respective positions and for water-proof sealing of the catheter at its tip.

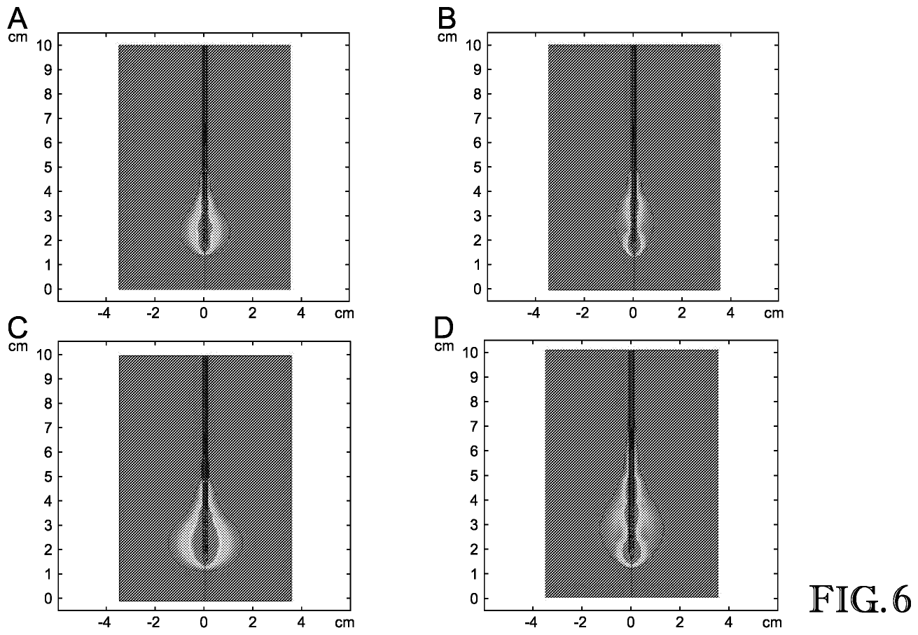

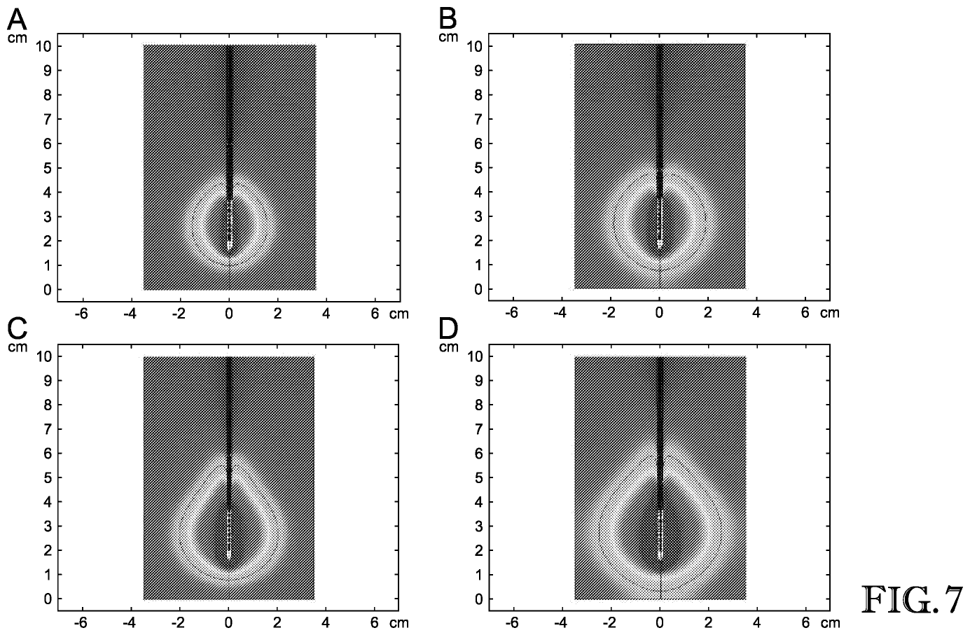

Simulations were performed to determine the electromagnetic heat losses and temperature profiles of the microwave applicator. FIG. 6 shows simulated electromagnetic heat losses of the microwave applicator at different power levels and times during an ablation treatment, namely electromagnetic heat loss of the applicator at 30 W and (A) 0s, (B) 10 min, and at 80 W and (C) 0s and (D) 10 min. FIG. 7 shows the coupled electromagnetic-thermal simulations that reveal the estimated shape of the ablation zone. The temperature profiles are shown at power 30 W and (A) 5 min, (B) 10 min, and at power 80 W and (C) 5 min and (D) 10 min. The black solid contour represents the 60° C. isotherm.

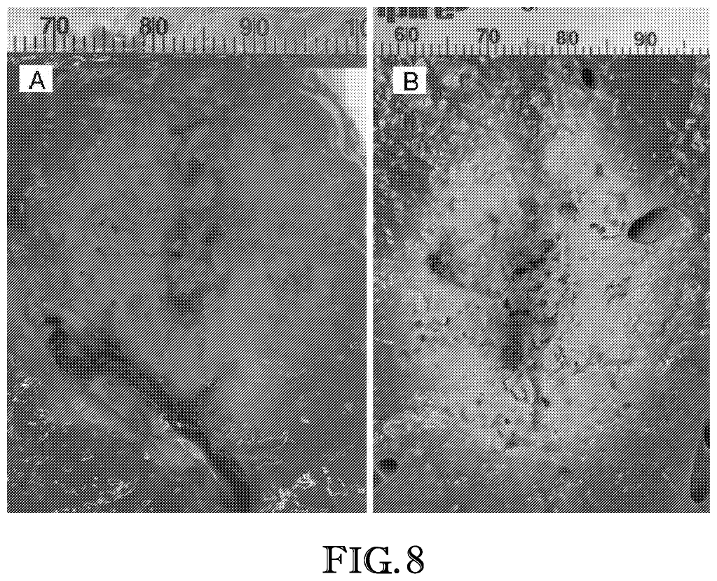

Results of the coupled electromagnetic-thermal simulations were verified experimentally on ex vivo liver tissue and at two power/time combinations. The resulting ablation zones are shown in FIG. 8 (ablation zones achieved in ex vivo liver tissue with (A) 80 W power applied for 10 min and a 40% duty cycle with a period of 10 s and (B) 60 W power applied for 10 min).

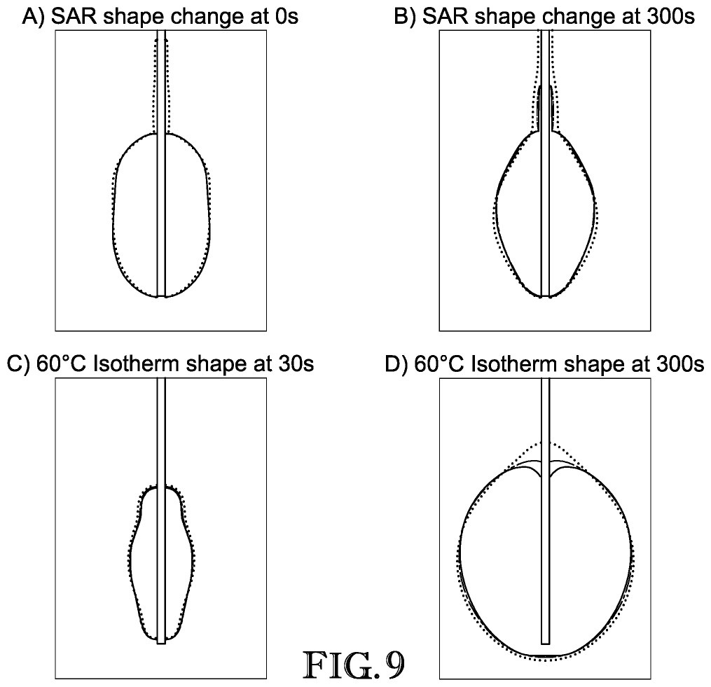

FIG. 9 illustrates the impact of cooling water temperature on antenna performance and depicts the predicted shape of the ablation zone as a function of cooling water temperature. Power absorption profiles are shown at (A) t=0 seconds and (B) t=300 seconds. Temperature profiles for the 60° C. isotherm are shown at (C) 30 seconds and (D) 300 seconds. The solid, dash-dot, and dotted lines represent the use of cooling water at 20° C., 40° C., and 50° C., respectively.

The use of water as the material on the outer surface of the choke further provides a means for tailoring a radiation pattern of the antenna. For example, when treating lesions that are oblong or with one axis relatively longer than others, it may be desirable to generate ablation zones with a longer length. The relative permittivity of water changes as a function of temperature. Thus, by adjusting coolant flow parameters, the shape of the ablation zone can be adjusted. Using a lower pump flow rate, or using higher temperature coolant water, can yield higher water temperatures within the catheter, and thus modulate the relative permittivity of the material on the outer surface of the third conductor.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

As used herein, the term “includes” may refer to an item that includes something as a part thereof or is entirely made up of that something.

Although the above description presents features of preferred embodiments of the present invention, other preferred embodiments may also be created in keeping with the principles of the invention. Such other preferred embodiments may, for instance, be provided with features drawn from one or more of the embodiments described above. Yet further, such other preferred embodiments may include features from multiple embodiments described above, particularly where such features are compatible for use together despite having been presented independently as part of separate embodiments in the above description.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventors hereby state their intent to rely on the Doctrine of Equivalents to determine and assess the reasonably fair scope of the present invention as pertains to any apparatus not materially departing from but outside the literal scope of the invention as set forth in the following claims.

1. An electrosurgical device for ablative treatment comprising:
an elongate catheter comprising proximal and distal catheter ends, the catheter distal end being configured for insertion into a body comprising the target tissue for ablation;
a transmission cable at least partially located within the catheter and comprising a cable proximal end configured to be connected to a power source for generating microwave power and a cable distal end, the transmission cable comprising at least one inner conductor and an outer conductor, the inner and outer conductors being electrically isolated from each other by a dielectric material;
an antenna extending from the cable distal end and configured to emit microwave energy therefrom; and
a choke element surrounding at least a portion of the transmission cable, the choke element comprising choke distal and proximal ends, the choke element distal end being electrically connected to the outer conductor, the choke element proximal end being radially spaced from the outer conductor.

2. The electrosurgical device of claim 1, wherein the catheter is a multi-lumen catheter, the transmission cable being located within one of the lumens, at least one other lumen being configured to circulate a cooling fluid between the catheter proximal and distal ends.

3. The electrosurgical device of claim 1, wherein the antenna is a linear antenna selected from the group consisting of monopole, dipole, slot, and helical radiating antennae.

4. (canceled)

5. (canceled)

6. The electrosurgical device of claim 1, wherein the electrosurgical device comprises an insulative material positioned between the choke element and the outer conductor, except for an electrical connection between the choke element and outer conductor at the choke element's distal end.

7. The electrosurgical device of claim 1, wherein the electrosurgical device includes a doping element providing a radiopaque marker for identifying the location of the electrical connection during an imaging process, wherein the doping element includes a joint that provides the electrical connection between the choke element and the outer conductor.

8. (canceled)

9. The electrosurgical device of claim 1, wherein the choke element has a tubular shape and presents one or more openings along the length thereof, and wherein the choke distal end is electrically connected to the cable distal end.

10. The electrosurgical device of claim 1, wherein the antenna comprises a segment of the inner conductor surrounded by the dielectric material.

11. (canceled)

12. An electrosurgical device for tissue ablation comprising:
an elongate catheter comprising proximal and distal catheter ends, the catheter distal end being configured for insertion into a body comprising the tissue targeted for ablation;
a transmission cable at least partially located within the catheter and comprising a cable proximal end configured to be connected to a power source for generating microwave power and a cable distal end, the transmission cable comprising at least one inner conductor and an outer conductor, the inner and outer conductors being electrically isolated from each other by a dielectric material;
an antenna extending from the cable distal end and configured to emit microwave power therefrom; and
a plurality of spaced-apart, annular conductive elements positioned around the antenna, at least one of the annular conductive elements being electrically connected to the inner conductor and at least one other of the annular conductive elements being electrically isolated from the inner conductor.

13. The electrosurgical device of claim 12, wherein the at least one annular conductive element being electrically connected to the inner conductor comprises the most distal conductive element of the plurality of conductive elements.

14. The electrosurgical device of claim 12, wherein at least one of the plurality of annular conductive elements is electrically connected to the outer conductor, and wherein the at least one annular conductive element electrically connected to the outer connector comprises the most proximal conductive element of the plurality of conductive elements.

15. (canceled)

16. The electrosurgical device of claim 12, wherein the antenna is a linear antenna selected from the group consisting of monopole, dipole, and helical radiating elements.

17. The electrosurgical device of claim 12, wherein the plurality of annular conductive elements comprise coils wrapped around the dielectric material.

18. (canceled)

19. The electrosurgical device of claim 12, wherein the electrosurgical device further comprises a choke element surrounding at least a portion of the transmission cable, and wherein the choke element comprises choke distal and proximal ends, the choke element distal end being electrically connected to the outer conductor, and the choke element proximal end being radially spaced from the outer conductor.

20. (canceled)

21. The electrosurgical device of claim 12, wherein the electrosurgical device further comprises a tube located within the catheter, the tube and the catheter cooperating to define a flow path configured to circulate a cooling fluid between the catheter proximal and distal ends.

22. A method of ablating tissue within a body comprising:
inserting at least one electrosurgical device of claim 1 into the body containing the tissue to be ablated;
positioning the device antenna into or adjacent to the tissue to be ablated; and
activating the device thereby causing the antenna to emit electromagnetic power that is sufficiently strong to cause ablation of the tissue.

23. The method of claim 22, wherein activating the device for at least five minutes creates an ablation zone within the tissue having an axial ratio of from about 0.75:1 to about 1:0.75.

24. (canceled)

25. The method of claim 22, wherein the positioning step includes the step of visualizing at least part of the device antenna and the tissue via fluoroscopy imaging to determine the device antenna position relative to the tissue.

26. A method of ablating tissue within a body comprising:
positioning the device antenna into or adjacent to the tissue to be ablated;
activating the device thereby causing the antenna to emit electromagnetic power that is sufficiently strong to cause ablation of the tissue and define a predicted ablation zone; and
changing a shape of the predicted ablation zone by circulating a flow of cooling water along the device antenna.

27. The method of claim 26, wherein said changing step includes:
(a) the step of changing a temperature of the cooling water to change the shape of the predicted ablation zone, wherein said step of changing the cooling water temperature includes the step of cooling the temperature of the cooling water to shift a proximal end of the predicted ablation zone distally; or
(b) the step of changing the cooling water flow rate to change the shape of the predicted ablation zone.

28. (canceled)

29. (canceled)

30. The method of claim 26, said device antenna being located within an elongate catheter that presents first and second lumens extending between catheter proximal and distal ends, wherein the step of circulating the flow of cooling water through the device antenna includes the step of directing the flow of cooling water distally through the first lumen and proximally through the second lumen.
